(19) United States
(12) Patent Application Publication
(10) Pub. No.: US 2017/0258953 A1
(43) Pub. Date: Sep. 14, 2017

(54) ELECTRONEGATIVE FIBRE FOR USE IN THE HEALING OF WOUNDS

(71) Applicant: URGO RECHERCHE INNOVATION ET DEVELOPPEMENT

(72) Inventor: Jean-Marc Pernot, Dijon (FR)

(21) Appl. No.: 15/505,989

(22) PCT Filed: Sep. 7, 2015
(86) PCT No.: PCT/FR2015/052369

§ 371 Date: Feb. 23, 2017

(30) Foreign Application Priority Data
Sep. 8, 2014 (FR) 1458396

(51) Int. Cl. A61L 15/12

(52) U.S. Cl. CPC A61L 15/12

(57) ABSTRACT

A subject of the present invention is a material comprising at least one electronegative fiber having a negative charge density of between 0.01 and 5 mmol/g and a liquid absorption capacity of less than 9.5 g of physiological saline per g of fiber, for the use thereof in wound healing, and preferentially in wound cleaning. Another subject of the invention is a medical device, such as a dressing or a bandage, comprising such a material.

A subject of the present invention is a material comprising at least one electronegative fiber for the use thereof in the healing of wounds, and preferentially in the cleaning of wounds, especially of chronic wounds. Another subject of the invention is a medical device, such as a dressing or a bandage, comprising said material.

A dressing is a protective device making it possible to cover a wound located on the skin. A dressing may have several functions, which may or may not be combined, such as:
protecting the wound (from infection or irritation and isolating it from the external environment;
enabling better healing by maintaining a beneficial moist environment at the wound bed;
stopping minimal bleeding by compressing small blood vessels;
bringing together the edges of a wound;
absorbing exudates in order to preserve the edges of the wound and the perilesional skin.

Some types of dressing are therefore specifically developed to promote wound healing, while others do not have this purpose.

The natural healing of a wound takes place in three successive phases, each of these phases being characterized by specific, different cellular activities: the cleaning phase, the granulation phase and the epithelialization phase. Throughout the healing process, the wound produces fluid or viscous exudates which must, if possible, be absorbed by the healing dressing or evacuated by the latter to be guided towards a receptacle outside the wound (in the case of negative pressure therapy—NPT).

The natural cleaning abilities of the wound may be insufficient when there is a large trauma or when the patient is suffering from concomitant disorders, such as venous disorders or diabetes. In these cases, a considerable lengthening of the duration of the cleaning phase is observed, leading to chronic wounds which are difficult to treat, such as a leg ulcer.

In the case of wounds for which the natural cleaning process is insufficient, it is necessary to remove the fibrinous tissue without disrupting the granulation phase. The removal of this fibrinous tissue is commonly denoted by the term “assisted cleaning”, as opposed to natural cleaning.

Depending on the technique used, assisted cleaning can be classed as mechanical or surgical cleaning, enzymatic cleaning, autolytic cleaning or biological cleaning.

Mechanical or surgical cleaning is a rapid technique that consists of cutting away the fibrinous tissue, either using a lancet, forceps, scissors or a Brock curette, or by means of sophisticated apparatus using water jets under pressure or laser excision. This technique is performed at the patient's bed or in the surgical environment depending on the severity of the wound. However, this technique is often painful and can lead to bleeding and sometimes even a hemorrhage. It is then traumatic for the patient. It commonly also requires prior analgesic medication, which increases the treatment time.

Autolytic cleaning consists of placing absorbent materials on the wound, such as dressings or bandages, based on particular gelling fibers. The purpose of such a technique is either to be able to soften the fibrin to subsequently enable its removal by means of a curette by skilled medical or paramedical personnel, or to enable its removal by an action of attachment of the fibrin to the absorbent material, or optionally to combine these two methods in an optimal way.

The materials conventionally used to facilitate cleaning are needled nonwovens of alginate or carboxymethylcellulose gelling fibers.

The carboxymethylcellulose fibers gel on contact with the exudates, enabling effective softening of the fibrin, but considerably adversely affecting the cohesion of the nonwoven material comprising them, thereby preventing any attachment during removal of the material.

The alginate fibers also make it possible to soften the fibrin, thereby promoting its manual removal. Nonetheless, even though the alginate fibers do not gel as significantly on contact with the exudates as do the carboxymethylcellulose fibers, thereby preserving the cohesion of the nonwoven material comprising them, they do not have any properties of attachment of the fibrin during removal of the material.

A material enabling both softening of the fibrin and attachment of the latter during its removal, while preserving the cohesion thereof, would be an optimal compromise.

Document WO 2012/131263 precisely proposes using superabsorbent fibers, such as those sold by TOYOBO which have a liquid absorption capacity of 27.8 g of water per grain of fiber and a negative charge density of 12.3 mmol/g, in order to obtain a nonwoven which makes it possible to soften the fibrin and to promote its attachment during its removal. However, the superabsorbent properties of the fibers used may cause a phenomenon referred to as “gel blocking”, that is to say that the fibers swell in the presence of fluids containing water, until they form a compact gel which is sealed off from the exudates, no longer enabling the absorption and diffusion thereof. In order to avoid this phenomenon, application WO 2012/131263 proposes using a nonwoven comprising a mixture of non-absorbent thermal bonding fibers and superabsorbent fibers, with which a specific contact layer is associated. Such a product certainly has a good autolytic ability while preserving its cohesion during its removal and without generating any “gel blocking” phenomena, but remains complicated and costly to manufacture.

It would therefore be desirable to have a simple material which is easy to manufacture, for treating wounds, especially chronic wounds, which has a good autolytic cleaning ability, that is to say which enables an optimized action of attachment of the fibrin in order to reduce or eliminate the need for surgical procedures, without however adversely affecting the absorption and diffusion of exudates.

Surprisingly, the applicant discovered that it was possible to respond to these problems by means of a material based on an electronegative fiber having a specific fluid absorption capacity and a specific negative charge density.

Thus, according to a first aspect, a subject of the invention is a material comprising at least one electronegative fiber having a negative charge density of between 0.01 and 5 mmol/g and a liquid absorption capacity of less than 9.5 g of physiological saline per gram of fiber, for the use thereof in the healing of wounds, preferentially in the cleaning of wounds, and especially of chronic wounds.

According to a second aspect, another subject of the invention is a medical device, such as a dressing, especially a healing dressing, or a bandage, comprising such a material.

Indeed, the applicant has observed, that the material according to the invention or the medical device employing said material guarantees the necessary fibrin removal for good autolytic cleaning. Indeed, the inventors especially demonstrated that by using electronegative fibers having a specific fluid absorption capacity, it was possible to obtain a material attaching to fibrin and enabling its removal during the removal of the material, while promoting the absorption and diffusion of exudates.

The present invention thus makes it possible to optimize the healing of wounds, especially of chronic wounds, by proposing for the first time a material which simultaneously promotes autolytic cleaning and the absorption and diffusion of liquid exudates without causing a phenomenon of “gel blocking”.

Finally, the material of the invention also has the advantage of being cohesive and of not tearing when it is removed.

Electronegative Fiber

The material according to the present invention comprises at least one electronegative fiber having a negative charge density of between 0.01 and 5 mmol/g, preferably between 0.05 and 4 mmol/g, and more preferentially between 0,1 and 2 mmol/g.

The fibers used in the material of the invention also have a liquid absorption capacity of less than 9.5 g of physiological saline per gram of fiber, preferably ranging from 2 to 9.4 g of physiological saline per gram of fiber, and more preferentially from 3 to 9.3 g of physiological saline per gram of fiber.

The physiological saline within the meaning of the present application is a 0.9% solution of sodium chloride NaCl.

The fibers according to the invention may be non-absorbent when they have a liquid absorption capacity of approximately 0 g of physiological saline per gram of fiber, sparingly absorbent or absorbent when they have a liquid absorption capacity of strictly greater than 0 g of physiological saline per gram of fiber and less than 9.5 g of physiological saline per gram of fiber, They are therefore differentiated in particular from fibers classed as “superabsorbent”, having a liquid absorption capacity of greater than 20 g of water (or of saline solution such as physiological saline) per gram of fiber, such as the TOYOBO fibers used in application WO 2012/131263.

The absorption of physiological saline by a fiber or a textile comprising these fibers may for example be measured with physiological saline comprising 0.9% NaCl as described above by applying the procedures described in the Edana 440.1.99 methods.

The electronegative fiber according to the invention is preferably a polymer fiber, The polymer constituting the fiber may especially be chosen from polyesters, polyamides, polyolefins and copolymers thereof, acrylic polymers, polyurethane, polyacrylates and copolymers thereof, and cellulose polymers and mixtures thereof.

According to a preferred embodiment, the polymer constituting the electronegative fiber according to the present application is a cellulose polymer, preferably a cellulose derivative or viscose, or an acrylic polymer, preferably a copolymer of acrylonitrile and vinyl chloride.

A fiber having the desired negative charge density according to the present application may be obtained by incorporating particles having cation or polyelectrolyte exchange properties during the manufacture of said fiber.

The particles having cation or polyelectrolyte exchange properties may be introduced into the polymer matrix during the synthesis of the polymer, or during the spinning of the polymer by incorporating said particles or said polyelectrolytes into the formulation for supplying the spinning device.

According to a preferred embodiment, the electronegative fibers used in the material of the invention are obtained by incorporating ion exchange resins bearing sultanate groups and/or by incorporating polyelectrolytes bearing carboxylate groups into a viscose polymer matrix, such as those sold by Kelheim under the names Poseidon or Verdi. Patent application US 2006/0246285 describes such a manufacturing process.

Alternatively, the fiber having the desired negative charge density according to the present application may be obtained by chemical synthesis of a polymer having suitable ionic groups.

According to a preferred embodiment, the electronegative fibers used in the material of the invention are obtained by reacting a modacrylic polymer, and in particular an acrylonitrile/vinyl chloride copolymer, with an amine having an ion exchange group. Such fibers are especially sold by Kaneka under the trade names Kanecaron ion exchange fiber®. Patent application EP 2 703 556 describes such a manufacturing process.

Material

The material according to the invention may be in the torn of a woven or nonwoven material, a knit, a thread, a bundle or a cluster of fibers.

Preferably, the material according to the invention is in the form of a material preferably chosen from the list consisting of nonwovens, wovens or knits.

The fibers may preferably be assembled under conditions making it possible to obtain a textile material with a high basis weight, that is to say materials with a basis weight of greater than 75 g/m².

Contact Layer

According to one particular embodiment, and as long as this does not adversely affect its good fibrin attachment and cohesion properties, the material according to the invention may be partially covered with a contact layer on the face of the material which is intended to come into contact with the wound, said layer comprising openings enabling the passage of wound exudates.

Advantageously, the contact layer is said to be microadhesive, that is to say it makes it possible to temporarily affix the material of the invention to the wound. The assembly may then be removed without the structure of the wound or of the perilesional skin being adversely affected, so that said assembly is repositionable and facilitates nursing care. This temporary affixing can also assist the care personnel or the user in securing the material with other fixing means, for example in covering the material with a support means or an adhesive tape. In this case, the contact layer may be chosen such that it has an adhesive strength on a steel plate of between 0.5 and 100 cN/cm, preferably of between 5 and 40 cN/cm. This adhesive strength is measured according to the method EN 1939, in which a sample of contact layer 20 mm wide and 150 mm long is placed on a steel plate and in which, after 10 minutes, the adhesive strength is measured with a dynamometer at a pull rate of 100 mm/min at a 90° angle.

The contact layer may preferably be formed from a composition comprising an elastomer matrix and hydrocolloids, and in particular an elastomer matrix in which hydrocolloids are preferably homogeneously dispersed.

The proportion of hydrocolloids is preferably between 2 and 20% by weight of the weight of said composition.

The contact layer may in particular cover between 55 and 65% of the face of the casing that is intended to come into contact with the wound.

The contact layer preferably has a basis weight ranging from 110 to 500 g/m², preferably from 150 to 200 g/m².

The contact layer advantageously makes it possible not to stick to the wound and to avoid any pain on removal of the healing material. By maintaining a moist environment at the surface of the wound while avoiding contact with the nonwoven material, it improves healing. The incorporation of hydrocolloids gives the elastomer composition a hydrophilic nature and promotes the delivery of active agents capable of promoting the treatment of the wound.

Said composition comprises one or more elastomers chosen from poly(styrene-olefin-styrene) block polymers. The block copolymers used within the context of the invention are advantageously triblock copolymers of ABA type comprising two styrene thermoplastic end blocks A and an elastomer central block B which is an olefin, optionally combined with diblock copolymers of AB type comprising a styrene thermoplastic block A and an elastomer block B which is an olefin. The olefin blocks B of these copolymers may consist of unsaturated olefins such as for example isoprene or butadiene or of saturated olefins such as for example ethylene-butylene or ethylene-propylene.

In the case of a mixture of triblock copolymers ABA and of diblock copolymers AB, it will be possible to use commercial mixtures of triblock copolymers ABA and of diblock copolymers AB that are already available or to produce mixtures in any proportion chosen beforehand from two independently available products.

The triblock copolymers with an unsaturated central block are well known to those skilled in the art and are especially sold by Kraton Polymers under the name KRATON®□ D.

As examples of poly(styrene-isoprene-styrene) (abbreviated to SIS) copolymers, mention may thus be made of the products sold under the names KRATON®□ D1107 or KRATON®□ D1119 BT or else the products sold by Exxon Mobil Chemical under the name VECTOR® such as for example the product sold under the name VECTOR® 4113. An example of poly(styrene-butadiene-styrene) copolymers is the product sold under the name KRATON® D1102.

As examples of commercial mixtures of triblock copolymers ABA and of diblock copolymers AB in which B is isoprene, mention may be made of the products sold by Exxon Mobil Chemical under the name VECTOR® 4114.

All these copolymers based on isoprene or on butadiene generally have a styrene content of between 10% and 52% by weight relative to the total weight of said copolymer.

Within the context of the present invention, use will preferably be made of the poly(styrene-isoprene-styrene) (abbreviated to SIS) triblock block copolymers having a styrene content of between 14% and 52% and preferably of between 14% and 30% by weight relative to the weight of said poly(SIS).

Preferably, for producing the compositions of the present invention, use will be made of triblock block copolymers and in particular the product sold by Kraton Polymers under the name KRATON® D1119 BT.

The triblock copolymers having a saturated central block are also well known to those skilled in the art and are, for example, sold:
by Kraton Polymers under the name KRATON® G, and in particular under the name KRATON® G1651, KRATON® G1654 or KRATON® G1652 for poly(styrene-ethylene-butylene-styrene) (abbreviated to SEBS) block copolymers;
by Kuraray under the name SEPTON® for poly(styrene-ethylene-propylene-styrene) (abbreviated to SEPS) block copolymers.

As an example of commercial mixtures of triblock and diblock copolymers, mention may be made of the product sold by Kraton Polymers under the name KRATON® G1657, the olefin block of which is ethylene-butylene.

As an example of a particular mixture of triblock and diblock copolymers that can be produced within the context of the present invention, mention may be made of the mixture:
of a triblock SEBS, such as in particular the product sold by Kraton Polymers under the name KRATON® G1651; and
of a poly(styrene-olefin) diblock copolymer such as, in particular, the poly(styrene-ethylene-propylene) sold by Kraton Polymers under the name KRATON® G1702.

Within the context of the present invention, SEBS or SEPS triblock copolymers having a styrene content of between 25% and 45% by weight relative to the weight of said SEBS or SEPS will be preferred. Preferably, use will be made of triblock block copolymers and in particular the products sold by the company Kraton Polymers under the names KRATON® G1651 and KRATON® G1654.

Generally, the elastomer will be used in suitable amounts depending on the saturated or unsaturated nature of the olefin central block of the block copolymer. Thus, in the case of a triblock copolymer having an unsaturated central block it will be used in an amount of the order of 10% to 30% by weight, preferably of 10% to 20% by weight, relative to the total weight of the composition, In the case of a triblock copolymer having a saturated central block, it will be used in an amount of the order of 3% to 10% by weight, preferably of 4% to 7% by weight, relative to the total weight of the composition.

The term “hydrocolloid” or “hydrocolloid particles” is intended to mean here any compound customarily used by those skilled in the art for its ability to absorb aqueous liquids such as water, physiological saline or wound exudates.

As suitable hydrocolloids, mention may for example be made of pectin, alginates, natural vegetable gums such as, in particular, Karaya gum, cellulose derivatives such as carboxymethyl celluloses and the alkali metal salts thereof such as sodium or calcium, and also synthetic polymers based on acrylic acid salts, known under the name “superabsorbents”, such as, for example, the products sold by BASF under the name LUQUASORB® 1003 or by Ciba Specialty Chemicals under the name SALCARE® SC91 and also mixtures of these compounds.

Some of these superabsorbents, classed as “microcolloids” since they have a particle size of less than 10 micrometers, may of course be used within the context of the production of the composition.

The hydrocolloids preferred within the context of the present invention are the alkali metal salts of carboxymethylcellulose, and in particular sodium carboxymethylcellulose (CMC). The size of the hydrocolloid particles is for example between 50 and 100 microns, especially of the order of 80 microns.

The amount of hydrocolloids incorporated into the elastomer composition will advantageously be of the order of 2% to 20% by weight, preferably of 5% to 18% by weight, more preferably still of 8% to 18% by weight, more preferably still of 12% to 16% by weight, relative to the total weight of the elastomer composition. Hydrocolloids introduced in too large an amount into a perforated contact layer reduce the absorption capacity of a nonwoven based on superabsorbent fibers as the gel forms. Indeed, the high absorption capacity of the hydrocolloids leads to a swelling of the contact layer, so much so that the holes of the mesh may become blocked. The nonwoven no longer directly absorbs the exudates but absorbs the exudates present in the hydrocolloid absorbent layer, which reduces the absorption capacity of the composite material and creates problems of maceration.

According to one preferred embodiment, the contact layer may comprise one or more elastomers chosen from the poly(styrene-olefin-styrene) block polymers combined with one or more plasticizing compound(s) intended to improve their stretching, flexibility, extrudability or processing properties.

They will preferably be liquid compounds, compatible with the olefin central block of the block copolymers used.

Among the plasticizing compounds capable of being used for this purpose, mention may in particular be made of plasticizing mineral oils, irrespective of the nature of the central block. Mention may also be made of polybutenes—such as, for example, the products sold by BP Chemicals under the name NAPVIS® 10—or else of phthalate derivatives such as dioctyl phthalate or dioctyl adipate, when the central block is unsaturated.

Alternatively, it is also possible to use synthetic products based on liquid mixtures of saturated hydrocarbons such as, for example, the products sold by Total under the name GEMSEAL® and in particular the product GEMSEAL® 60 which is an isoparaffinic mixture derived from a completely hydrogenated petroleum cut. Use will preferably be made of these products with a triblock copolymer comprising a saturated central block.

Within the context of the present invention, use will preferably he made of plasticizing oils and in particular of mineral oils formed from compounds of paraffinic, naphthenic or aromatic nature or mixtures thereof in variable proportions.

Among the plasticizing oils that are particularly suitable, mention may be made of:
the products sold by Shell under the names ONDINA® and RISELLA® which consist of mixtures based on naphthenic and paraffinic compounds;
the products sold under the name CATENEX® which consist of mixtures based on naphthenic, aromatic and paraffinic compounds.

Particularly preferably, use will be made of a mineral plasticizing oil chosen from the products sold under the names ONDINA® 933 and ONDINA® 919.

These plasticizing compounds may be used in an amount of the order of 20% to 65% by weight, preferably of 30% to 50% by weight, relative to the total weight of the hydrocolloid elastomer composition.

According to one embodiment, these compositions are said to be adherent: they have the property of adhering to the skin without adhering to the wound. They comprise one or more compounds referred to as “tackifiers” such as those customarily used by those skilled in the art in the preparation of elastomer-based pressure-sensitive adhesives. For a detailed description of these products, reference may be made to the work by Donatas Satas “Handbook of Pressure Sensitive Technology”, 3rd Edition, 1999, pages 346 to 398.

Generally, it will be possible to use one (or more) tackifying product(s) which will be incorporated into the elastomer matrix in a proportion of the order of 1% to 50% by weight, relative to the total weight of the hydrocolloid elastomer composition, which will be determined as a function of the nature and of the relative proportion of the other constituents thereof, in order to achieve the desired microadhesive strength for the casing.

Preferably, the tackifying product(s) will represent from 10% to 45% by weight, and more preferably still from 15% to 40% by weight of the total weight of the hydrocolloid elastomer composition.

The tackifying products capable of being used within the context of the present invention will be able to be chosen from tackifying resins, low molecular weight polyisobutylenes or mixtures thereof.

Among the tackifying resins capable of being used according to the invention, mention may be made of modified terpene or polyterpene resins, rosin resins, hydrocarbon resins, mixtures of cyclic, aromatic and aliphatic resins, or mixtures of these resins.

Such products are sold, for example:
by Arakawa Chemical Industries under the name ARKON® P which are hydrogenated polycyclopentadiene resins;
by Exxon Chemical under the name ESCOREZ® and in particular the 5000 series of resins which are hydrogenated;
by Goodyear under the name WINGTACK®, and in particular WINGTACK® 86 which is a synthetic resin formed from C5/C9 copolymers or WINGTACK® 10 which is a resin based on synthetic polyterpene;
by the company Hercules under the name KRISTALEX® and in particular KRISTALEX® 3085 which is a resin based on α-methylstyrene.

Generally, in order to avoid the problems of coloring and stability of unsaturated resins, the use of hydrogenated resins, in particular with triblock copolymers having a saturated central block, will be preferred since they are much more compatible with the latter than WINGTACK type unsaturated resins that are essentially used with triblock copolymers having an unsaturated central block.

Among the latter, use will preferably be made of ESCOREZ® resins of the 5000 series and most particularly the ESCOREZ® 5380 resin.

The tackifying resins may be used alone or as a mixture with other tackifying products, preferably in a proportion of 10% to 50% by weight, and more particularly of 15% to 40% by weight, relative to the total weight of the composition.

Among the low molecular weight polyisobutylenes capable of being used as tackifying products, mention may be made of the polyisobutylenes having a molecular weight of the order of 40 000 to 80 000 daltons, such as for example the products sold by BASF under the name OPPANOL® and in particular the products sold under the names OPPANOL® B12 and OPPANOL® B15 or by Exxon Chemical under the name Vistanex and in particular the LM-MH grade.

These polyisobutylenes will be able to be used alone or as a mixture with other tackifiers in combination with triblock copolymers having an unsaturated central block, Their proportion will be able to vary, in this case, from 5% to 30% by weight, and more particularly from 8% to 15% by weight, relative to the total weight of the composition.

Active Agents

Various compounds may also be added to the material of the present invention, such as, in particular, active agents or adjuvants commonly used in the field of wound treatment or in the pharmacological field.

The material may contain active agents that have a favorable role in the treatment of the wound. These active agents may preferably induce or promote wound healing. Other active agents may also be used within the context of the invention, such as, for example, bactericidal or bacteriostatic agents, antiseptics, painkillers or local anesthetics, anti-inflammatories, antipruritics, calmatives, hydrating agents, antioxidants, depigmenting agents and mixtures thereof.

Generally, these active agents may be chosen from:
active agents promoting healing, such as Retinol, Vitamin A, Vitamin E, N-acetyl-hydroxyproline, Centella asiatica extracts, papain, silicones, thyme, niaouli, rosemary and sage essential oils, hyaluronic acid, Allantoin, -Hema'tîte (gattefossé), Vitamin C, TEGO Pep 4-17 (evonik), Toniskin (silab), Collageneer (Expanscience), Timecode (Seppic), Gatuline skin repair (gattefossé), Panthenol, PhytoCellTec Alp Rose (Mibelle Biochemistry), Erasyal (libragen), Serilesine (Lipotec), Heterosides of Talapetraka (Bayer), Stoechiol (codif), Macarose (Sensient), Dermaveil (Ichimaru Pharcos), Phycosaccaride AI (Codif), growth factors, metformin, synthetic polysulfated oligosaccharides having 1 to 4 monosaccharide units, such as in particular sucrose octasulfate potassium salt (known by the abbreviation KSOS), sold in the product Urgotul® Start by Laboratoires Urgo;
bactericidal or bacteriostatic agents such as polymyxin B, penicillins (amoxycillin), clavulanic acid, tetracyclines, minocycline, chlortetracycline, aminoglycosides, amikacin, gentamicin, neomycin, probiotics, silver salts such as for example silver sulfate, silver chloride, silver nitrate, silver sulfadiazine, quaternary ammoniums, polyhexamethylene biguanide and chlorhexidine;
antiseptics, such as thiomersal, eosin, chlorhexidine, phenylmercuric borate, aqueous hydrogen peroxide solution, Dakin's solution, triclosan, biguanide, hexamidine, thymol, Lugol's solution, iodinated povidone, merbromin, benzalkonium chloride, benzethonium chloride, ethanol or isopropanol;
painkillers or local anesthetics such as paracetamol, codeine, dextropropoxyphene, tramadol, morphine and its derivatives, or corticoids and derivatives;
anti-inflammatories, such as glucocorticoids, nonsteroidal anti-inflammatories, aspirin, ibuprofen, ketoprofen, flurbiprofen, diclofenac, aceclofenac, ketorolac, meloxicam, piroxicam, tenoxicam, naproxen, indomethacin, naproxcinod, nimesulide, celecoxib, etoricoxib, parecoxib, rofecoxib, valdecoxib, phenylbutazone, niflumic acid or mefenamic acid;
depigmenting agents, such as kojic acid (Kojic Acid SL®—Quimasso (Sino Lion)), arbutin (Olevatin®—Quimasso (Sino Lion)), the mixture of sodium palmitoyl propyl and of European water lily extract (Sepicalm®—Seppic) or undecylenoylphenylalanine (Sepiwhite®—Seppic);
antipruritics: hydrocortisone, enoxolone, diphenhydramine, locally applied anti-H1 antihistamine;
moisturizing active agents, such as Xpermoist (Lipotec), hyaluronic acid, urea, fatty acids, glycerol, waxes or Exossine (Unipex);
UV-screening agents, such as Parsol MCX or Parsol 1789;
calmatives, such as camomile, bisabolol, xanthalene, glycyrrhetinic acid, tanactin (CPN) or Calmiskin (Silab);
antioxidants, such as vitamin E.

According to a preferred embodiment, the active agents which may be introduced into the material according to the present invention are preferably chosen from active agents which promote healing, anti-inflammatories and mixtures thereof.

“Active agent which promotes healing” is intended to mean any active agent capable of acting favorably at any stage of the healing process via any sort of interaction, that is to say via any interaction of biological, chemical or physical nature, with the wound in contact with which said active agent is applied.

More particularly, the active agents which may be introduced into the material according to the present invention or the contact layer which may be associated therewith are preferably chosen from synthetic polysulfated oligosaccharides having 1 to 4 monosaccharide units, such as in particular sucrose octasulfate potassium salt, aspirin, silver sulfate, silver sulfadiazine, metformin and mixtures thereof.

Generally, the material according to the present invention may comprise active agents in an amount of from 0.01 to 20% by weight, preferably from 1 to 15% by weight and more preferably still from 2 to 10% by weight, relative to the total weight of the material containing them.

The material of the invention may also contain adjuvants, among which mention may be made of dyestuffs, fillers, odor absorbers or trappers, pH regulators, microcapsules or microspheres that may optionally contain active agents, vaseline, polymers or surfactants making it possible to optimize the gelling rate, wettability or release of the active agents from the material.

Medical Device

Another subject of the invention is a medical device comprising the material promoting the healing described above. “Medical device” is intended to mean an item of equipment used by humans for preventing, controlling, treating or relieving a disease or injury.

Such a medical device may especially be a dressing, especially a healing dressing, a bandage, or a composite material for packing wounds, especially cavity wounds.

The present invention is illustrated in more detail in the following non-limiting example.

### EXAMPLE

Preparation of the Materials According to the Invention.

The following 3 materials according to the invention were prepared:

Fibers, of the Cationic Exchange Fiber model, sold by Kaneka under the name Kanecaron ion exchange fiber® were used, and they were shaped into a 97 g/m² nonwoven material. The fibers constituting said nonwoven have a negative charge density of 1.5 mmol/g and a liquid absorption capacity of 3 g of physiological saline per gram of fiber.

Fibers, of the Cationic Exchange Fiber type model, sold by Kaneko under the name Kanecaron ion exchange fiber® were also used, and they were shaped into a 109 g/m² nonwoven material. These fibers have a negative charge density of 1.5 mmol/g and a liquid absorption capacity of 6.3 g of physiological saline per gram of fiber.

Finally, Poseidon 3.3 dtex/40 mm fibers sold by Kelheim were used in the form of a cluster of non-transformed fibers directly resulting from the spinning process. These fibers have a negative charge density of 1,55 mmol/g and a liquid absorption capacity of 9.1 g of physiological saline per gram of fiber.

Test of In Vitro Removal of the Fibrin Matrix:

The fibrin matrices were prepared according to the protocol described by Brown in the publication “Fibroblast migration in fibrin gel matrices”, Am J. Pathol, 1993, 142: 273-283.

The components and the procedure which were used are as follows:

The following were solubilized at 37° C.:
5 ml of an aqueous solution comprising 50 mmol of HEPES (Sigma-Aldrich catalog)
15 mg of fibrinogen from human plasma (Sigma-Aldrich catalog)
5 mmol of CaCl₂.

50 μl of thrombin and 100 NIH of human plasma (Sigma-Aldrich catalog) were added to the solution thus prepared.

Everything was placed in a Petri dish and left to incubate at 37° for 24 hours.

The fibrin matrix is formed after 24 hours.

According to a first test protocol, a sample of material chosen from one of the 3 samples described above is deposited on the matrix at room temperature and then removed immediately.

According to a second test protocol, a sample of material chosen from one of the 3 samples described above is deposited on the matrix at room temperature with a weight of 500 g for 30 seconds then removed.

On removal, for each tested sample of material according to the invention, according to either one of the two protocols followed, it is observed that the fibrin had detached from the Petri dish and had transferred in a single piece to the surface of the material which was removed; this was the case for each test protocol conducted.

In parallel, comparative tests were carried out.

Thus, a nonwoven compress-type product consisting of carboxymethylcellulose fibers and sold under the name Aquacel® was tested for in vitro removal of a fibrin matrix according to the second protocol described above.

The carboxymethylcellulose fibers have a negative charge density of 1.5 mmol/g and a physiological saline absorption capacity of 24.1 g per gram of fiber.

It is observed that the fibrin matrix is not detached from the Petri dish. Moreover, when the material is placed in contact with an exudate simulator (such as a liquid solution), said material consisting of carboxymethylcellulose fibers gels, thereby becoming barely cohesive and thus making it impossible in all cases to remove said material without tearing.

Another nonwoven compress-type product, this time consisting of alginate fibers and sold under the name Algostéril®, was tested for in vitro removal of a fibrin matrix according to the second protocol described above.

The alginate fibers have a negative charge density of 5.10 mmol/g and a physiological saline absorption capacity of 12.8 g per gram of fiber.

It is observed here that the fibrin matrix is not detached from the Petri dish, even though said product is removed in a single piece. The fibrin attachment is zero.

1. A material comprising at least one electronegative fiber having a negative charge density of between 0.01 and 5 mmol/g and a liquid absorption capacity of less than 9.5 g of physiological saline per gram of fiber, for the use thereof in wound healing.

2. The material as claimed in claim 1, wherein said material is formulated as a wound cleaning composition.

3. The material of claim 1, wherein the electronegative fiber has a negative charge density of between 0.05 and 4 mmol/g.

4. The material of claim 1, wherein the electronegative fiber is a polymer fiber, the polymer constituting the fiber being chosen from polyesters, polyamides, polyolefins and copolymers thereof, polyurethane, acrylic polymers, polyacrylates and copolymers thereof, and cellulose polymers and mixtures thereof.

5. The material of claim 1, wherein the polymer constituting the electronegative fiber is a cellulose polymer, preferably a cellulose derivative or viscose, or an acrylic polymer, preferably a copolymer of acrylonitrile and vinyl chloride.

6. The material as of claim 1, wherein the electronegative fiber is obtained either by incorporating particles having cation or polyelectrolyte exchange properties during the manufacture of said fiber, or by chemical synthesis of a polymer having suitable ionic groups.

7. The material as of claim 1, wherein the electronegative fiber is obtained by incorporating ion exchange resins bearing sulfonate groups and/or by incorporating polyelectrolytes bearing carboxylate groups into a viscose polymer matrix, or by reacting a modacrylic polymer, and in particular an acrylonitrile/vinyl chloride copolymer, with an amine having an ion exchange group.

8. The material of claim 1, wherein said material is in the form of a nonwoven, a woven or a knit.

9. The material of claim 1, wherein said material has a basis weight of greater than 75 g/m².

10. The material of claim 1, wherein said material is partially covered with a contact layer on the face of the material which is intended to come into contact with the wound, said layer comprising openings enabling the passage of wound exudates.

11. The material of claim 1, wherein said material contains active agents promoting wound healing.

12. A medical device comprising a material of claim 1, wherein said device being a dressing or a bandage.

13. A medical device comprising a material of claim 1, wherein said device is a composite material for packing cavity wounds.

14. The material of claim 1, wherein the electronegative fiber has a negative charge density of between 0.1 and 2 mmol/g.
